(19) United States
(12) Patent Application Publication
(10) Pub. No.: US 2014/0212345 A1
(43) Pub. Date: Jul. 31, 2014

(54) ISOLATOR

(71) Applicant: Panasonic Healthcare Co., Ltd., Ehime (JP)

(72) Inventors: Eiji KOUNO, Aichi (JP); Hironobu SEKINE, Gunma (JP); Yasuhiko YOKOI, Saitama (JP); Koichi KOBAYASHI, Tochigi (JP)

(73) Assignee: Panasonic Healthcare Co., Ltd., Ehime (JP)

(21) Appl. No.: 14/229,281

(22) Filed: Mar. 28, 2014

Related U.S. Application Data
(63) Continuation of application No. PCT/JP2012/075212, filed on Sep. 28, 2012.

(30) Foreign Application Priority Data
Sep. 30, 2011 (JP) 2011-218150

(51) Int. Cl. C12M 1/00; C12M 1/12

(52) U.S. Cl. CPC C12M 41/14; C12M 37/00
USPC 422/565

(57) ABSTRACT

An isolator includes: a working chamber for conducting work on cells, the working chamber including an opening on a front face thereof; a transparent plate member, made of a resin, through which an interior of the working chamber can be viewed, the plate member being mounted to the working chamber so as to close the opening; and a working chamber sterilization device configured to sterilize the interior of the working chamber by supplying hydrogen peroxide gas into the working chamber, and thereafter discharge the hydrogen peroxide gas in the working chamber, the plate member having at least a face, facing the working chamber, formed to have a water absorption rate equal to or smaller than a predetermined value.

### CROSS-REFERENCE TO RELATED APPLICATIONS

This is a continuation application of International Patent Application No. PCT/JP2012/075212 filed Sep. 28, 2012, which claims the benefit of priority to Japanese Patent Application No. 2011-218150 filed Sep. 30, 2011. The full contents of the International Patent Application are incorporated herein by reference.

### BACKGROUND

1. Technical Field

The present disclosure relates to an isolator.

2. Description of the Related Art

Isolators have been developed that are capable of conducting work, such as cell culture and inspection, in a sealed working chamber that has been brought closer to an aseptic condition by killing microorganisms, bacteria, and the like.

In the case of conducting work on cells and the like using an isolator, a working chamber is sterilized beforehand. Sterilization of an inside of a working chamber is performed by, first, supplying sterilizing gas, such as hydrogen peroxide gas, into the working chamber, and thereafter carrying out aeration in which air containing the sterilizing gas in the working chamber is exchanged with outside air. Various techniques relating to sterilization processes using such hydrogen peroxide gas have been developed (for example, see

Note that, in the present specification, to bring a state closer to an aseptic condition by killing microorganisms, bacteria, and the like is referred to as sterilization.

The isolator is a cabinet whose interior can be made an airtight space, and includes a window, through which the interior thereof can be viewed, and a working glove. This window is usually made of a resin, such as acrylic, polycarbonate (PC), polyamide (PA), or polyethylene terephthalate (PET). These resins have absorbency with respect to hydrogen peroxide. Thus, when hydrogen peroxide gas is supplied to sterilize an interior space, hydrogen peroxide is partially absorbed by the aforementioned window member. Therefore, even if aeration is carried out, the absorbed hydrogen peroxide is not easily discharged, which causes a lengthy time period for aeration.

The present disclosure provides an isolator capable of reducing the time period for aeration.

### SUMMARY

An isolator according to an aspect of the present disclosure, includes: a working chamber for conducting work on cells, the working chamber including an opening on a front face thereof; a transparent plate member, made of a resin, through which an interior of the working chamber can be viewed, the plate member being mounted to the working chamber so as to close the opening; and a working chamber sterilization device configured to sterilize the interior of the working chamber by supplying hydrogen peroxide gas into the working chamber, and thereafter discharge the hydrogen peroxide gas in the working chamber, the plate member having at least a face, facing the working chamber, formed to have a water absorption rate equal to or smaller than a predetermined value.

Other features of the present disclosure will become apparent from descriptions of the present specification and of the accompanying drawings.

### BRIEF DESCRIPTION OF THE DRAWINGS

For more thorough understanding of the present disclosure and advantages thereof, the following description should be read in conjunction with the accompanying drawings, in which:

FIG. 1 is an exemplary overall configuration diagram of an isolator according to an embodiment of the present disclosure;

FIG. 2 is an exemplary diagram illustrating a working chamber of an isolator according to an embodiment of the present disclosure;

FIG. 3A is an exemplary cross-sectional diagram of a viewing window according to an embodiment of the present disclosure;

FIG. 3B is an exemplary cross-sectional diagram of a viewing window according to an embodiment of the present disclosure;

FIG. 3C is an exemplary cross-sectional diagram of a viewing window according to an embodiment of the present disclosure;

FIG. 3D is an exemplary cross-sectional diagram of a viewing window according to an embodiment of the present disclosure;

FIG. 3E is an exemplary cross-sectional diagram of a viewing window according to an embodiment of the present disclosure;

FIG. 4 is an exemplary diagram illustrating hydrogen peroxide residual levels of materials according to an embodiment of the present disclosure;

FIG. 5 is an exemplary diagram illustrating water absorption rates of materials according to an embodiment of the present disclosure; and

FIG. 6 is an exemplary diagram illustrating a relationship between hydrogen peroxide residual level and water absorption rate in materials according to an embodiment of the present disclosure.

### DETAILED DESCRIPTION

At least the following details will become apparent from descriptions of the present specification and of the accompanying drawings.

### Configuration of Isolator

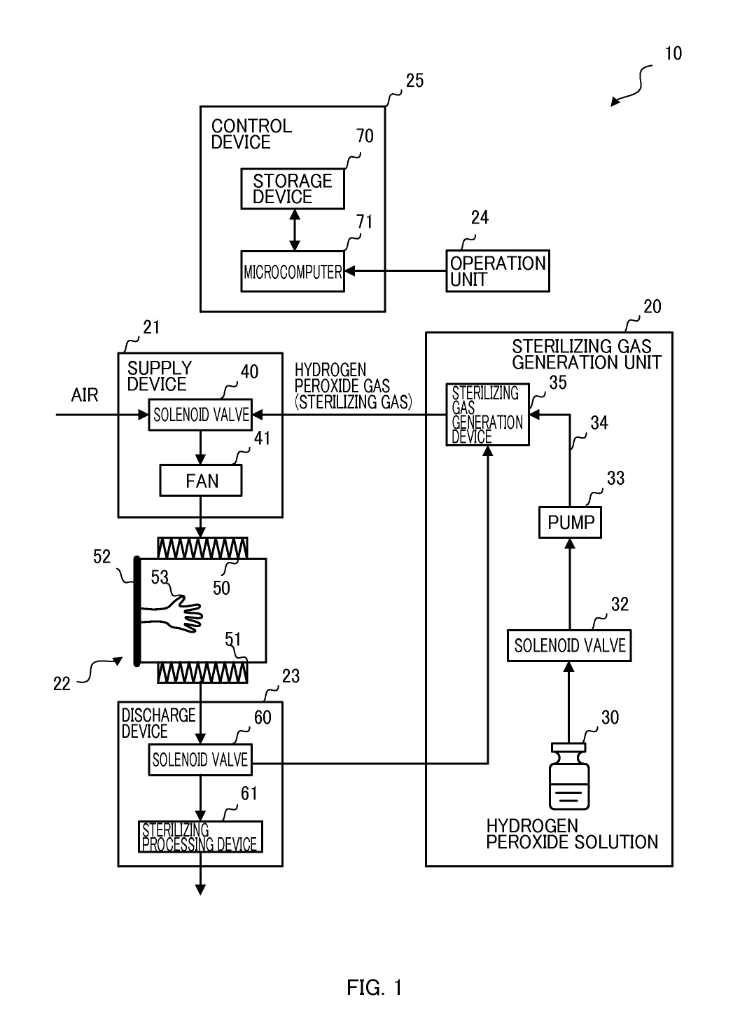

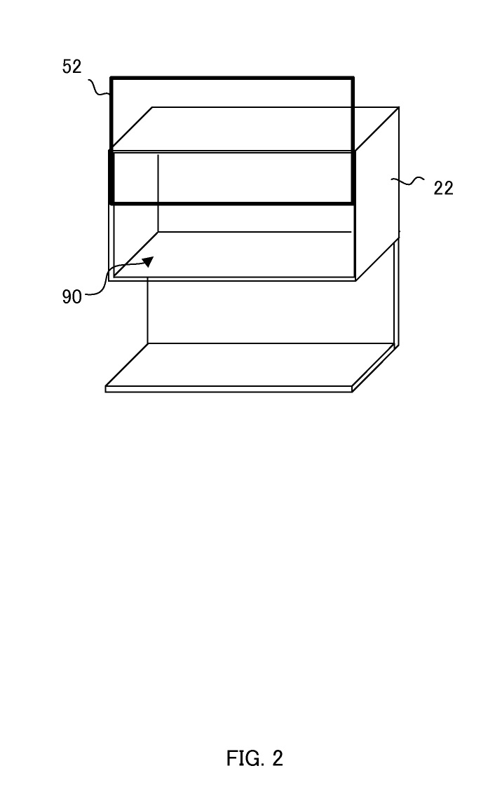

A configuration of an isolator 10 according to an embodiment of the present disclosure will be described with reference to FIG. 1 and FIG. 2. The isolator 10 is a device configured to conduct work on cells and the like in a sterilized environment, and includes a sterilizing gas generation unit 20, a supply device 21, a working chamber 22, a discharge device 23, an operation unit 24, and a control device 25.

Note that the sterilizing gas generation unit 20, the supply device 21, the discharge device 23, and the control device 25 are equivalent to a working chamber sterilization device.

The sterilizing gas generation unit 20 is a device unit configured to generate sterilizing gas, and includes a tank 30, a solenoid valve 32, a pump 33, a pipe 34, and a sterilizing gas generation device 35. Note that operations of the solenoid valve 32, the pump 33, and the sterilizing gas generation device 35 are controlled by the control device 25.

The tank 30 is configured to store hydrogen peroxide solution (a solution containing dissolved hydrogen peroxide (H₂O₂)).

The solenoid valve 32 is a solenoid valve configured to connect the tank 30 to the pump 33 under control of the control device 25.

The pump 33 is configured to pump up the hydrogen peroxide solution from the tank 30 and supply the solution through the pipe 34 to the sterilizing gas generation device 35.

The sterilizing gas generation device 35 is configured to generate hydrogen peroxide gas, which is sterilizing gas, on the basis of the hydrogen peroxide solution supplied from the pump 33, and supply the hydrogen peroxide gas to the supply device 21 with air which is a carrier gas.

The supply device 21 is a device configured to supply the supplied hydrogen peroxide gas or air outside the isolator 10 to the working chamber 22, and includes a solenoid valve 40 and a fan 41.

The solenoid valve 40 is configured to supply hydrogen peroxide gas or the outside air to the fan 41 under control of the control device 25. The fan 41 is configured to supply the hydrogen peroxide gas or air supplied from the solenoid valve 40 to the working chamber 22.

The working chamber 22 is a cabinet, made of metal, in a substantially rectangular parallelepiped shape having a space for working on cells in an interior thereof. For example, the working chamber 22 is made of stainless steel (SUS). The working chamber 22 is provided with air filters 50 and 51, a viewing window 52, and a working glove 53.

As illustrated in FIG. 2, the working chamber 22 includes, on a front face thereof, an opening 90 for bringing cells and the like into the interior thereof. Further, the transparent viewing window 52, through which the interior of the working chamber 22 can be viewed, is mounted in an openable and closable manner to the opening 90.

The viewing window 52 is formed using a plate member that is transparent, break-resistant and lightweight, and is made of a resin such as acrylic, polycarbonate (PC), polyamide (PA), or polyethylene terephthalate (PET), for example.

Returning to FIG. 1, the air filter 50 is a filter configured to remove dust and the like that are contained in the hydrogen peroxide gas or the air supplied from the fan 41. The air filter 51 is a filter configured to remove dust and the like that are contained in the gas and the like discharged from the working chamber 22. Note that, for example, an HEPA (High Efficiency Particulate Air) filter is used for the air filters 50 and 51.

The working glove 53 is attached to an opening (not shown) that is provided at the viewing window 52 so that a worker can conduct work on cells and the like in the working chamber 22 with the viewing window 52 closed. Note that, with the viewing window 52 closed, the working chamber 22 is sealed.

The discharge device 23 is a device configured to discharge gas, such as hydrogen peroxide gas, and air, from the working chamber 22, and includes a solenoid valve 60 and a sterilizing processing device 61.

The solenoid valve 60 is configured to supply the gas outputted from the air filter 51 to either the sterilizing processing device 61 or the sterilizing gas generation device 35 under control of the control device 25. Note that, in the case where an output from the solenoid valve 60 is supplied to the sterilizing gas generation device 35, the gas in the working chamber 22 results in being circulated.

The sterilizing processing device 61 includes, for example, a catalyst, and is configured to subject the gas outputted from the solenoid valve 60 to detoxifying and sterilizing processes and output the gas to the outside of the isolator 10.

The operation unit 24 is an operation panel or the like through which a user sets an operation of the isolator 10. Operation results of the operation unit 24 are transmitted to the control device 25, and the control device 25 controls blocks in the isolator 10 on the basis of the operation results.

The control device 25 is a device configured to integrally control the isolator 10, and includes a storage device 70 and a microcomputer 71.

The storage device 70 is configured to store program data to be executed by the microcomputer 71 and various data. The microcomputer 71 is configured to implement various functions by executing the program data stored in the storage device 70.

### Process of Sterilizing Working Chamber

A process of sterilizing the interior of the working chamber 22 is performed, for example, by outputting an instruction to sterilize the interior of the working chamber 22 from the operation unit 24 to the control device 25.

When the instruction to sterilize the interior of the working chamber 22 is outputted from the operation unit 24, the microcomputer 71 executes a predetermined program, to cause the sterilizing gas generation unit 20 to generate hydrogen peroxide gas. Then, the microcomputer 71 controls the solenoid valve 40, the fan 41, and the solenoid valve 60, so that the hydrogen peroxide gas is circulated through the sterilizing gas generation device 35, the supply device 21, the working chamber 22, and the discharge device 23. As such, the hydrogen peroxide gas is supplied into the working chamber 22, thereby killing microorganisms, microbes, and the like in the working chamber 22.

After supplying the hydrogen peroxide gas for a predetermined time period, the microcomputer 71 stops the sterilizing gas generation unit 20. Then, the microcomputer 71 controls the solenoid valve 40, the fan 41, and the solenoid valve 60, so that aeration is carried out in which air outside the isolator 10 is sent from the supply device 21 into the working chamber 22 and also the air containing the hydrogen peroxide gas in the working chamber 22 is discharged from the discharge device 23.

Thereafter, when the hydrogen peroxide gas is sufficiently discharged from the interior of the working chamber 22, the microcomputer 71 stops the solenoid valve 40, the fan 41, and the solenoid valve 60, and makes the interior of the working chamber 22 in a sealed state. Thus, the process of sterilizing the interior of the working chamber 22 is completed.

Here, as described above, the viewing window 52 is made of a resin such as acrylic, polycarbonate (PC), polyamide (PA), or polyethylene terephthalate (PET). Although details will be described later, these resins have absorbency with respect to hydrogen peroxide.

Thus, in the case that the hydrogen peroxide gas is supplied into the working chamber 22 and the hydrogen peroxide is absorbed into the inside of the viewing window 52, aeration is required to be carried out over a long period of time until the hydrogen peroxide absorbed into the inside of the viewing window 52 has been discharged to the outside of the viewing window 52.

In this regard, the viewing window 52 according to an embodiment of the present disclosure is formed to have an index value, indicative of absorbency with respect to hydrogen peroxide, equal to or smaller than a predetermined value, as will be described in detail below.

Thus, the isolator 10 employing the viewing window 52 according to an embodiment of the present disclosure can reduce the time period required for aeration.

### Structure of Viewing Window 52

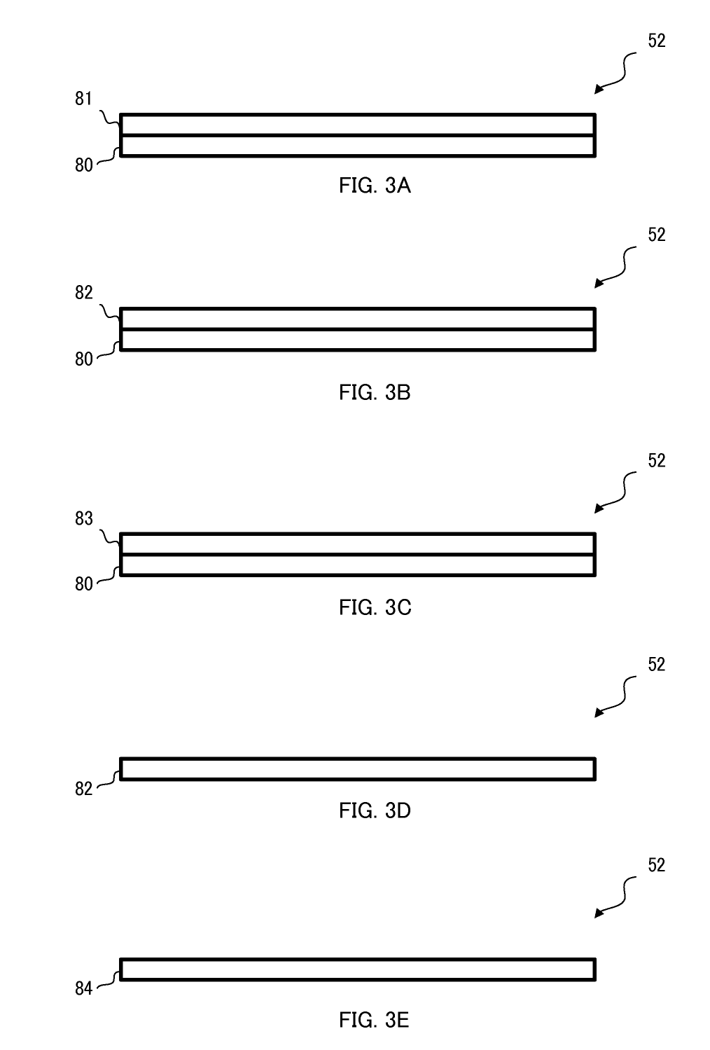

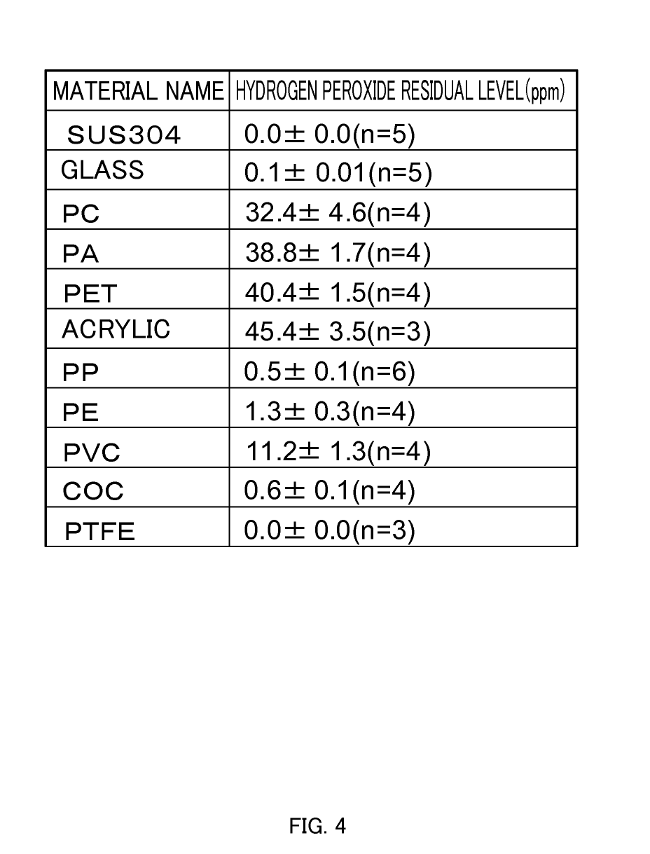

As a structure of the viewing window 52 according to an embodiment of the present disclosure, at least first to fifth embodiments are possible. The structures of the viewing window 52 according to the embodiments will be described with reference to FIGS. 3A to 3E and FIG. 4.

FIGS. 3A to 3E illustrate cross-sectional diagrams of the viewing window 52 according to the first to fifth embodiments, respectively. Further, FIG. 4 illustrates results of measuring index values indicative of absorbency of various materials with respect to hydrogen peroxide.

### Absorbency with Respect to Hydrogen Peroxide

The index values indicative of absorbency with respect to hydrogen peroxide illustrated in FIG. 4 are obtained by carrying out actual measurements according to the following procedure.

First, a sample, which is to be subjected to a measurement of absorbency with respect to hydrogen peroxide, is selected, and is stored in the sealed isolator 10. Then, the sample is exposed to the hydrogen peroxide gas at a predetermined concentration for a predetermined time period. Thereafter, aeration is carried out for a predetermined time period. When carrying out aeration, the maximum concentration of the hydrogen peroxide gas obtained by vaporization from the sample is measured.

The aforementioned measurements are carried out on the predetermined number of samples (the number indicated by “n” in FIG. 4) with respect to the materials listed in FIG. 4. Then, the average value of the maximum concentrations with respect to each of the materials is acquired, which is referred to as hydrogen peroxide residual concentration (ppm).

Therefore, the greater the numerical value of the hydrogen peroxide residual level of the material listed in FIG. 4 is, that is to say, the greater the index value indicative of absorbency with respect to hydrogen peroxide is, the greater the absorbency of the material with respect to hydrogen peroxide is.

### <First Embodiment>

The viewing window 52 according to a first embodiment of embodiments of the present disclosure is formed, as illustrated in FIG. 3A, by adhering a transparent film 81 to a transparent first plate member 80.

The viewing window 52 is mounted to the opening 90 of the working chamber 22 in a direction in which a surface of the film 81 is exposed to the interior of the working chamber 22.

The first plate member 80 is a transparent plate member made of a resin, for example, acrylic, polycarbonate (PC), polyamide (PA), or polyethylene terephthalate (PET).

By using the plate member made of the aforementioned resin as the first plate member 80 configuring the viewing window 52 according to an embodiment of the present disclosure, the viewing window 52 can be made more lightweight and break-resistant, as compared with the case of using glass, thereby improving operability and safety.

The film 81 is a transparent film made of, for example, polypropylene (PP), polyethylene (PE), tetrafluoroethylene (PTFE), or cyclic olefin copolymer (COC).

As illustrated in FIG. 4, all the hydrogen peroxide residual levels of these polypropylene (PP), polyethylene (PE), tetrafluoroethylene (PTFE), and cyclic olefin copolymer (COC) are equal to or smaller than 5.0 ppm, and are smaller than the hydrogen peroxide residual levels of acrylic, polycarbonate (PC), polyamide (PA), and polyethylene terephthalate (PET). That is to say, the absorption thereof with respect to hydrogen peroxide is quite small.

Thus, as the viewing window 52 according to an embodiment of the present disclosure, the aforementioned film 81 is adhered to the aforementioned first plate member 80, and the viewing window 52 is mounted to the opening 90 such that a surface of the film 81 is exposed to the interior of the working chamber 22, thereby being able to suppress to a low level an amount of the hydrogen peroxide absorbed by the viewing window 52 when sterilizing the interior of the working chamber 22.

### <Second Embodiment>

The viewing window 52 according to a second embodiment of the embodiments of the present disclosure is formed, as illustrated in FIG. 3B, by adhering a transparent second plate member 82 to the transparent first plate member 80.

The viewing window 52 is mounted to the opening 90 of the working chamber 22 in a direction in which a surface of the second plate member 82 is exposed to the interior of the working chamber 22.

The first plate member 80 is a transparent plate member made of a resin, similarly to the first embodiment, and is made of, for example, acrylic, polycarbonate (PC), polyamide (PA), or polyethylene terephthalate (PET).

By using the plate member made of the aforementioned resin as the first plate member 80 configuring the viewing window 52 according to an embodiment of the present disclosure, the viewing window 52 can be made more lightweight and break-resistant, as compared with the case of using glass, thereby improving operability and safety.

The second plate member 82 is a transparent plate member made of, for example, polypropylene (PP), polyethylene (PE), tetrafluoroethylene (PTFE), or cyclic olefin copolymer (COC).

Similarly to the first embodiment, as illustrated in FIG. 4, all the hydrogen peroxide residual levels of these polypropylene (PP), polyethylene (PE), tetrafluoroethylene (PTFE), and cyclic olefin copolymer (COC) are equal to or smaller than 5.0 ppm, and are smaller than the hydrogen peroxide residual levels of acrylic, polycarbonate (PC), polyamide (PA), and polyethylene terephthalate (PET).

Thus, as the viewing window 52 according to an embodiment of the present disclosure, the aforementioned second plate member 82 is adhered to the aforementioned first plate member 80, and the viewing window 52 is mounted to the opening 90 such that a surface of the second plate member 82 is exposed to the interior of the working chamber 22, thereby being able to suppress to a low level an amount of hydrogen peroxide absorbed by the viewing window 52 when sterilizing the interior of the working chamber 22.

### <Third Embodiment>

The viewing window 52 according to a third embodiment of the embodiments of the present disclosure is formed, as illustrated in FIG. 3C, by forming a transparent coating film 83 on the transparent first plate member 80.

The viewing window 52 is mounted to the opening 90 of the working chamber 22 in a direction in which a surface of the coating film 83 is exposed to the interior of the working chamber 22.

The first plate member 80 is a transparent plate member made of a resin, similarly to the first embodiment, and is made of, for example, acrylic, polycarbonate (PC), polyamide (PA), or polyethylene terephthalate (PET).

By using the plate member made of the aforementioned resin as the first plate member 80 configuring the viewing window 52 according to an embodiment of the present disclosure, the viewing window 52 can be made more lightweight and break-resistant, as compared with the case of using glass, thereby improving operability and safety.

The coating film 83 is a transparent film formed by a coating containing at least either a transition element or a transition element compound.

The transition element is an element from Group 3 to Group 11 in the periodic table, for example, iron, copper, manganese, titanium, or the like. The transition element compound is, for example, iron chloride, copper chloride, manganese dioxide, titanium dioxide, or the like.

The transition element and the transition element compound are known to decompose hydrogen peroxide into water and oxygen. Thus, a surface of the first plate member 80 is coated with a coating containing the transition element and/or the transition element compound, and the coating film 83 is kept exposed to the interior of the working chamber 22, thereby decomposing the hydrogen peroxide gas in the working chamber 22 that has been in contact with the viewing window 52 into water and oxygen. Thus, the hydrogen peroxide residual level in the coating film 83 containing the transition element and/or the transition element compound is remarkably decreased.

Therefore, as the viewing window 52 according to an embodiment of the present disclosure, the aforementioned coating film 83 is formed onto the aforementioned first plate member 80, and the viewing window 52 is mounted to the opening 90 such that a surface of the coating film 83 is exposed to the interior of the working chamber 22, thereby making it possible for hydrogen peroxide not to be easily absorbed by the viewing window 52 when sterilizing the interior of the working chamber 22.

Note that, as the transition element and the transition element compound to be contained in the coating film 83, the aforementioned iron, copper, manganese, titanium, iron chloride, copper chloride, manganese dioxide, and titanium dioxide are particularly excellent in cost, ease in availability, and the like.

Further, glass can be used as the coating film 83. Although glass does not have a function to decompose hydrogen peroxide, it does not have absorbency with respect to hydrogen peroxide. Thus, when sterilizing the interior of the working chamber 22, it becomes possible to suppress an amount of hydrogen peroxide absorbed by the viewing window 52 to substantially zero.

### <Fourth Embodiment>

The viewing window 52 according to a fourth embodiment of the embodiments of the present disclosure is formed, as illustrated in FIG. 3D, by the transparent second plate member 82.

The second plate member 82 is formed by a transparent plate member made of polypropylene (PP), polyethylene (PE), tetrafluoroethylene (PTFE), or cyclic olefin copolymer (COC), as described in the second embodiment.

By mounting the viewing window 52 of the fourth embodiment to the opening 90 of the working chamber 22, a surface of the second plate member 82 is exposed to the interior of the working chamber 22.

By using the plate member made of the aforementioned resin as the second plate member 82 configuring the viewing window 52 according to an embodiment of the present disclosure, the viewing window 52 can be made more lightweight and break-resistant, as compared with the case of using glass, thereby improving operability and safety.

Further, as illustrated in FIG. 4, all the hydrogen peroxide residual levels of polypropylene (PP), polyethylene (PE), tetrafluoroethylene (PTFE), and cyclic olefin copolymer (COC) are equal to or smaller than 5.0 ppm.

Thus, the viewing window 52 according to an embodiment of the present disclosure is configured with the aforementioned second plate member 82, and the viewing window 52 is mounted to the opening 90 such that a surface of the second plate member 82 is exposed to the interior of the working chamber 22, thereby being able to suppress to a low level an amount of hydrogen peroxide absorbed by the viewing window 52 when sterilizing the interior of the working chamber 22.

Further, film-adhering work, coating work, plate-member-bonding work, and the like when manufacturing the viewing window 52 can be omitted, and a film, a coating film, and a plate member will not come off.

### <Fifth Embodiment>

The viewing window 52 according to a fifth embodiment of the embodiments of the present disclosure is formed, as illustrated in FIG. 3E, by a transparent third plate member 84.

The third plate member 84 is formed by mixing at least either a transition element or a transition element compound into a transparent plate member that is made of acrylic, polycarbonate (PC), polyamide (PA), polyethylene terephthalate (PET), polypropylene (PP), polyethylene (PE), tetrafluoroethylene (PTFE), or cyclic olefin copolymer (COC).

By mounting the viewing window 52 of the fifth embodiment to the opening 90 of the working chamber 22, a surface of the aforementioned third plate member 84 is exposed to the interior of the working chamber 22.

By using the plate member made of the aforementioned resin as the third plate member 84 configuring the viewing window 52 according to an embodiment of the present disclosure, the viewing window 52 can be made more lightweight and break-resistant, as compared with the case of using glass, thereby improving operability and safety.

As described in the third embodiment, the transition element and the transition element compound are known to decompose hydrogen peroxide into water and oxygen. Thus, a surface of the third plate member 84 containing the transition element and/or the transition element compound is kept exposed to the interior of the working chamber 22, thereby decomposing the hydrogen peroxide gas in the working chamber 22 that has been in contact with the viewing window 52 into water and oxygen. Therefore, the hydrogen peroxide residual level in the coating film 83 containing the transition element and/or the transition element compound is remarkably decreased.

Thus, the viewing window 52 according to an embodiment of the present disclosure is configured with the aforementioned third plate member 84, and the viewing window 52 is mounted to the opening 90 such that a surface of the third plate member 84 is exposed to the interior of the working chamber 22, thereby making it possible for hydrogen peroxide not to be easily absorbed by the viewing window 52 when sterilizing the interior of working chamber 22.

Further, film-adhering work, coating work, plate-member-bonding work, and the like when manufacturing the viewing window 52 can be omitted, and a film, a coating film, a plate member will not come off.

### Relationship with Absorbency

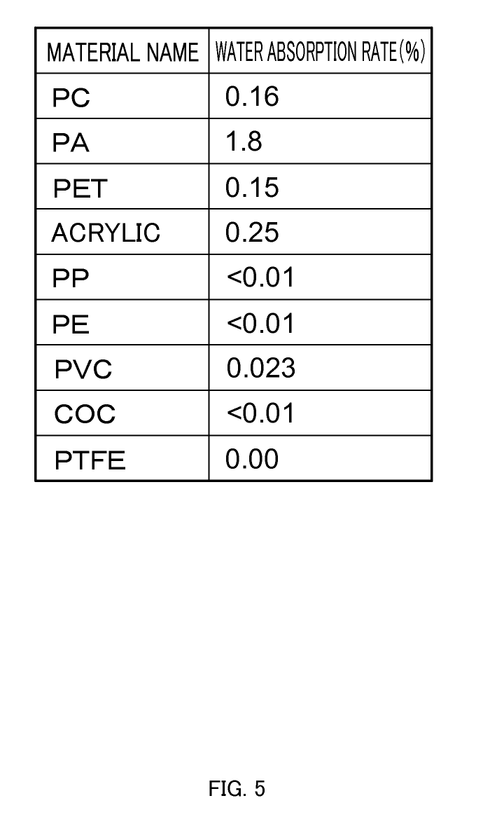

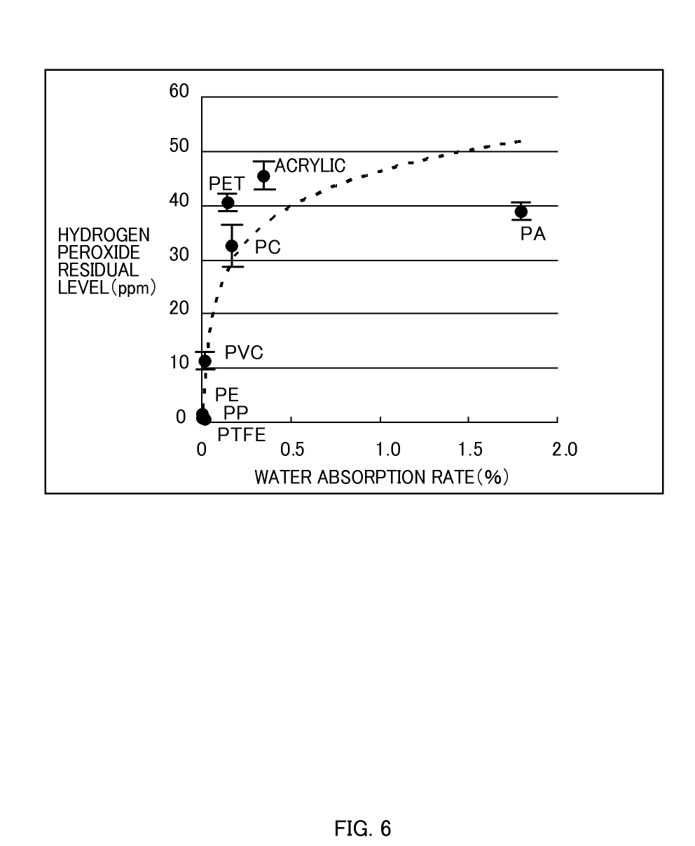

Note that FIG. 5 illustrates water absorption rates of materials. Further, FIG. 6 illustrates a relationship between the water absorption rates of the materials listed in FIG. 5 and residual levels of hydrogen peroxide solution in the materials listed in FIG. 4.

As indicated by a dashed line in FIG. 6, there is a correlation between the water absorption rate and the hydrogen peroxide residual level. Then, the film 81, the second plate member 82, the coating film 83, and the third plate member 84 are formed using a material with a hydrogen peroxide residual level equal to or smaller than 20 ppm (water absorption rate equal to or smaller than 0.1%), thereby obtaining preferable results. Further, it is preferable to use a material with a residual level equal to or smaller than 5 ppm (water absorption rate equal to or smaller than 0.01%).

Hereinabove, the isolator 10 according to an embodiment of the present disclosure has been described. According to the isolator 10 in an embodiment of the present disclosure, when sterilizing the interior of the working chamber 22 by supplying hydrogen peroxide gas into the working chamber 22, an amount of hydrogen peroxide absorbed by the viewing window 52 can be suppressed to a low level, thereby being able to reduce the time period required for aeration in which hydrogen peroxide gas is discharged from the working chamber 22. This enables efficient conduct of work on a larger amount of cells.

The above embodiments of the present disclosure are simply for facilitating the understanding of the present disclosure and are not in any way to be construed as limiting the present disclosure. The present disclosure may variously be changed or altered without departing from its spirit and encompass equivalents thereof.

What is claimed is:

1. An isolator comprising:
a working chamber for conducting work on cells, the working chamber including an opening on a front face thereof;
a transparent plate member, made of a resin, through which an interior of the working chamber can be viewed, the plate member being mounted to the working chamber so as to close the opening; and
a working chamber sterilization device configured to sterilize the interior of the working chamber by supplying hydrogen peroxide gas into the working chamber, and thereafter discharge the hydrogen peroxide gas in the working chamber,
the plate member having at least a face, facing the working chamber, formed to have a water absorption rate equal to or smaller than a predetermined value.

2. The isolator according to claim 1, wherein
the plate member is formed by adhering one of a transparent film and a transparent plate member to at least the face facing the working chamber, the transparent film and the transparent plate member being formed to have a water absorption rate equal to or smaller than a predetermined value.

3. The isolator according to claim 2, wherein
the one of the film and the plate member is made of one of polypropylene, polyethylene, tetrafluoroethylene, and cyclic olefin copolymer.

4. The isolator according to claim 1, wherein
the plate member is made of one of polypropylene, polyethylene, tetrafluoroethylene, and cyclic olefin copolymer.

5. The isolator according to claim 1, wherein
the plate member is formed by coating at least the face facing the working chamber with a coating formed to have a water absorption rate equal to or smaller than a predetermined value.

6. The isolator according to claim 5, wherein
the coating is formed to contain at least one of a transition element and a transition element compound.

7. The isolator according to claim 1, wherein
the plate member is formed to contain at least one of a transition element and a transition element compound.

8. The isolator according to claim 6, wherein
the transition element includes at least one of iron, copper, manganese, and titanium, and the transition element compound includes at least one of iron chloride, copper chloride, manganese dioxide, and titanium dioxide.

9. The isolator according to claim 7, wherein
the transition element includes at least one of iron, copper, manganese, and titanium, and the transition element compound includes at least one of iron chloride, copper chloride, manganese dioxide, and titanium dioxide.

10. The isolator according to claim 1, wherein
the water absorption rate is equal to or smaller than 0.01.

11. The isolator according to claim 2, wherein
the water absorption rate is equal to or smaller than 0.01.

12. The isolator according to claim 5, wherein
the water absorption rate is equal to or smaller than 0.01.
